(19) United States
(12) Patent Application Publication
(10) Pub. No.: US 2007/0164126 A1
(43) Pub. Date: Jul. 19, 2007

(54) Volatile liquid disseminating apparatus

(76) Inventors: Colin Brown, Bracknell Berkshire (GB); Guy Edward Naish, Bichester Oxfordshire (GB); Kishen Gohil, New Maiden Surrey (GB)

Correspondence Address: CURATOLO SIDOTI CO., LPA, 24500 CENTER RIDGE ROAD, SUITE 280, CLEVELAND, OH, 44145, US

(21) Appl. No.: 10/569,892

(22) PCT Filed: Aug. 20, 2004
(86) PCT No.: PCT/CH04/00528

§ 371 Date: Dec. 4, 2006

(30) Foreign Application Priority Data
Sep. 2, 2003 (GB) 0320514.3

(51) Int. Cl. A24F 25/00; A61L 9/04

(52) U.S. Cl. 239/34; 239/47; 239/55

(57) ABSTRACT

An apparatus for the dissemination of volatile liquid, such as a fragrance, into an atmosphere comprises a reservoir (1) containing volatile liquid (2) and at least one liquid transfer member (12) that is adapted to transfer liquid from the reservoir to the atmosphere. The transfer member is separated from the liquid, prior to putting the apparatus into operation, by a wall (5) having at least one breakable section (7), there being present at least one breaker element (11) that, when the apparatus is put into operation, is capable of breaking through the breakable section on being urged into contact therewith, thereby exposing the transfer member to the liquid. The transfer member preferably comprises capillary channels.

This invention relates to an apparatus for disseminating a volatile liquid into an atmosphere.

Many commercially-available apparatus for the dissemination into an atmosphere of volatile liquids, such as fragrances and insecticides, comprise a reservoir of volatile liquid and, extending therefrom, a liquid transfer member, typically a porous wick. In a more recent development, it has been proposed to replace the wick with an external capillary member, that is, a member bearing external channels of capillary dimensions. Such a member has been described in U.S. Pat. No. 4,913,350. Such members have the advantage of avoiding the undesirable fractionating effect that occurs particularly with multi-component fragrances, with one component evaporating into the atmosphere before the others. However, apparatus utilising this technology are prone to leakage during transport, and this has been a major factor in the lack of acceptance of this otherwise superior transfer member technology.

It has now been found that a new design of apparatus can overcome many or even all the problems of the art. The invention therefore provides an apparatus for the dissemination of volatile liquid into an atmosphere, the apparatus comprising a reservoir containing volatile liquid and at least one liquid transfer member that is adapted to transfer liquid from the reservoir to the atmosphere, the transfer member being separated from the liquid, prior to putting the apparatus into operation, by a wall having at least one breakable section, there being present at least one breaker element that, when the apparatus is put into operation, is capable of breaking through the breakable section on being urged into contact therewith, thereby exposing the transfer member to the liquid.

The invention additionally provides a method for disseminating controllably from a reservoir into an atmosphere a volatile liquid by providing in the reservoir a transfer member adapted to convey liquid from the reservoir to the atmosphere, the transfer member being separated from the liquid by a wall in which there is provided at least one breakable section, and causing dissemination when desired by urging a breaker element capable of breaking through the breakable section into breaking contact with that section, thus exposing the transfer member to the liquid.

The apparatus comprises the following essential elements:
transfer member
a wall separating the transfer member from the liquid, the wall having therein at least one breakable section
at least one breaker element capable of breaking through the breakable section in the wall and exposing the transfer member to the liquid
means for urging the element through the breakable section.

The reservoir can be any suitable reservoir of any suitable size and material. It has an orifice through which the transfer member extends to transfer liquid to the atmosphere.

The transfer member can be any suitable transfer member. It may, for example be a porous substance capable of absorbing and transferring liquid, typified by the fibrous and non-fibrous materials used for making wicks known to the art for the dissemination of such materials as fragrances and insecticides to the atmosphere. However, for the purposes of this invention, capillary channels are the preferred transfer member, and the description of the invention will henceforth be made solely with reference to this embodiment. It should nevertheless be borne in mind that porous materials are useful in this invention and that their use is not excluded.

Capillary channels are known as means for the transfer of a volatile liquid from a reservoir to an atmosphere, and are described, for example, in U.S. Pat. No. 4,913,350. They are typically V-shaped channels of dimensions of from 0.1-0.5 mm wide at the top, 0.1-0.5 mm deep with the “V” angle of the channel being 10-25 degrees, and they may be provided by any suitable means, such as moulding, engraving or cutting in any suitable material, for example, glass, ceramic, mineral or plastics materials.

The capillary channels extend downwards below the level of the liquid in the reservoir, but are prevented from making contact with the liquid by a wall. This wall has, at least adjacent to the liquid, at least one breakable portion, for example, a place where the wall is thinner. Although this breakable portion (and the corresponding element that breaks through the breakable portion) is referred to in the singular, the plural is also meant. Indeed, it is preferable that the wall have a number of such places, typically from 3 to 6. The wall may be any suitable shape, but is preferably cylindrical, with the breakable portion or portions running down the wall parallel to the cylindrical axis.

The apparatus additionally comprises at least one breaker element adapted to break through the breakable portion when urged to do so. Preferably the number of such elements is equal to that of the breakable portions, and they are arranged such that all elements break through all breakable portions essentially simultaneously. The breaker element may be any suitable element of any suitable material for breaking through the breakable portion, and the skilled person will readily be able to ascertain what is necessary in each case. Such an element will naturally have an appropriate shape, rigidity and strength relative to the breakable portion to break through the breakable portion.

In a typical arrangement, the breaker element is part of a cylinder that can be rotated, and rotation (for example, by twisting a cap) will bring it into contact with the breakable portion. The urge to break through may be provided by any suitable means. One way is by means of a ramp (such as a cam profile) placed opposite each breakable portion. Such an apparatus will typically have, in transverse cross-section, the appearance of a number of concentric rings, as outer fixed ring the wall with its breakable portions, as middle rotatable ring a ring with breaker elements and as inner fixed ring, a ring bearing outwardly-projecting cam profiles, with a cam profile placed opposite each breakable portion. Thus, the rotation of the middle ring will bring the breaker elements against the ramps cam profiles, which will urge them into contact with and then through the breakable portions of the wall to the liquid.

In an alternative version of the embodiment previously described, the outer and middle rings may be fixed, with the breaker elements positioned opposite the breakable portions, and the inner ring with the cam profiles can rotate, such that it pushes the breaker elements against and through the breakable portions.

The versions hereinabove described are not in any way limiting on the scope of the invention and the skilled person will readily be able to realise many other apparatus that lie within the scope of the invention, by the application of ordinary skill in the art.

The apparatus is so configured that this breaking of the breakable portions will bring the volatile liquid into contact with the capillary channels. This may be achieved by any convenient placing of the capillary channels with respect to the breakable portions, but in an especially preferred embodiment, the channels are incorporated in the breaker elements themselves. In one variant, the breaker elements have the form of elongate, vertically-orientated (with respect to the apparatus in use) members having a U-shaped cross-section, the U facing outwards towards the breakable portions and the bottom of the U comprising the channels. Preferably, these U-shaped members are sized such that they fit exactly into the breakable portions of the wall. Thus, when they break through the wall, the capillary channels are brought directly into contact with the liquid and the liquid can escape from the reservoir only via the channels. This provides a substantial degree of leakproofness.

There are many variations of this invention that are not described herein, but which fall within the scope of the invention, and the skilled person can readily comprehend these. The invention is further described with reference to a preferred embodiment, which is given only by way of example and is not limiting on the scope of the invention in any way. This embodiment is described in the drawings.

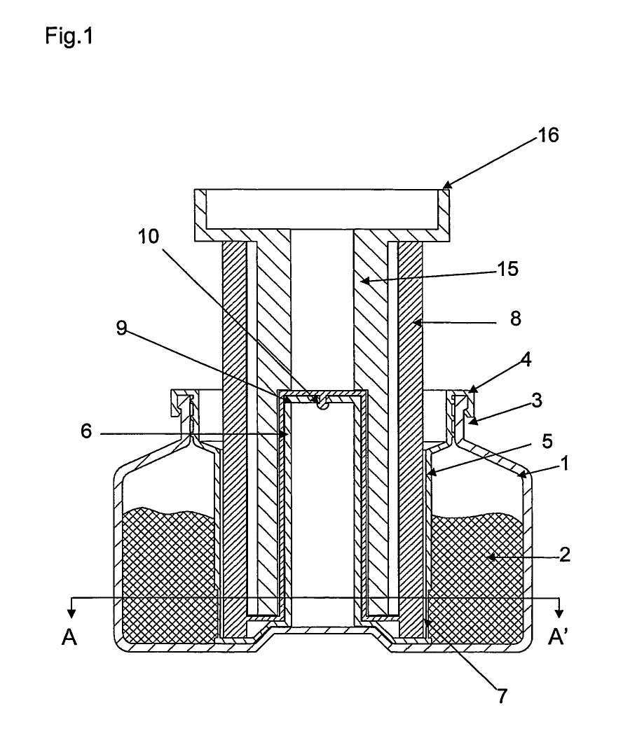

FIG. 1 is a longitudinal cross-section through a preferred embodiment of the invention.

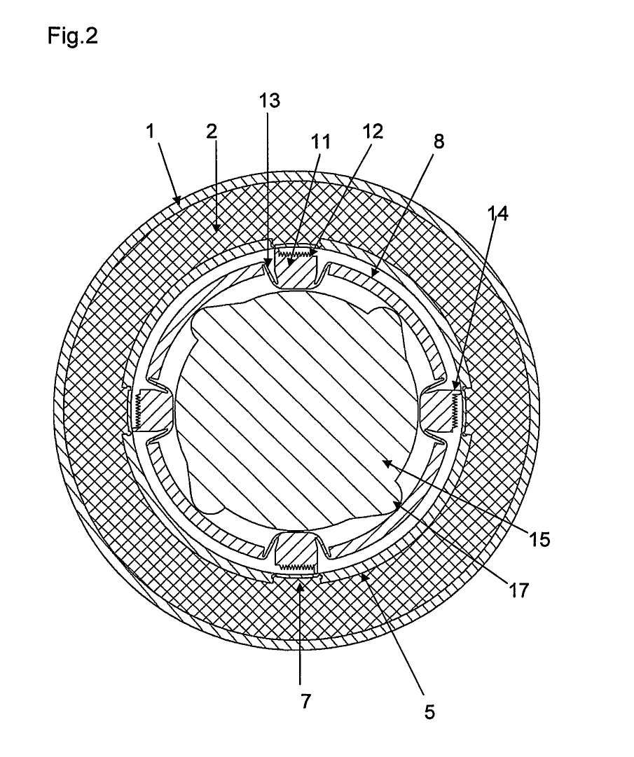

FIG. 2 is a simplified transverse cross-section through the mechanism of the embodiment along the line AA′.

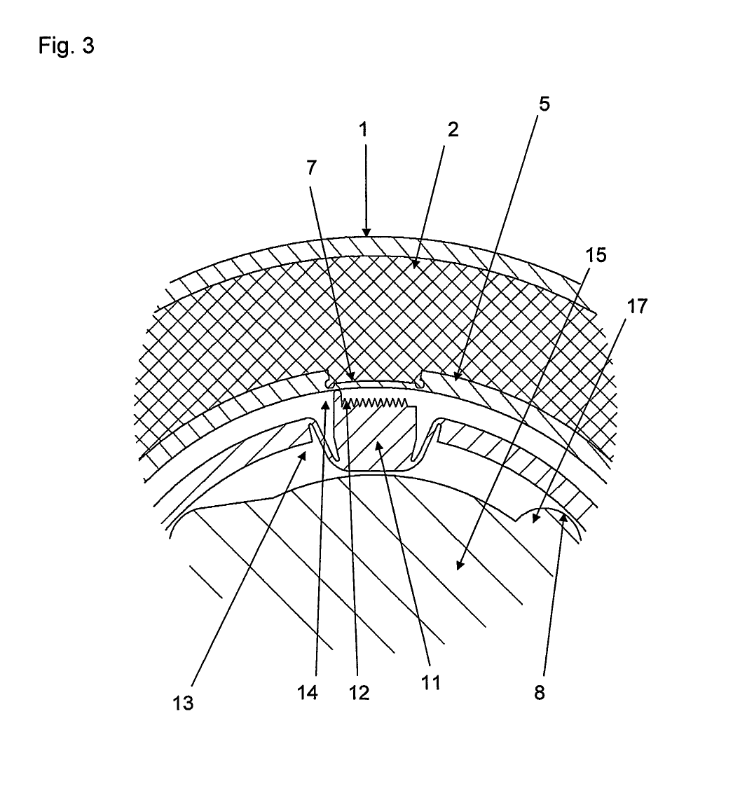

FIG. 3 is an enlargement of a portion of the embodiment of FIG. 1.

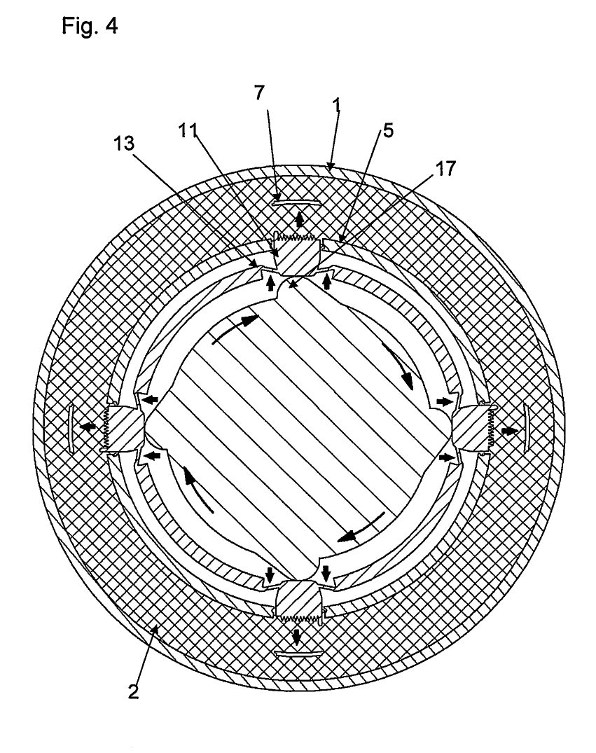

FIG. 4 is a version of FIG. 2, showing the actuation of the embodiment of FIGS. 1-3.

In the Figures, a reservoir 1 contains a volatile liquid 2. The reservoir has the form of a bottle with a neck 3. Fitting into the neck and fixed to it is an insert 4, which extends downwards from the neck to the bottom of the reservoir in the form of a cylindrical wall 5. The wall 5 is closed at the bottom, and extending upwardly from this closure is a coaxial cylindrical re-entrant portion 6. This insert is liquid-tight and therefore defines within the reservoir a proportion of the volume of the reservoir having no liquid. At the base of the wall 5 and spaced equidistantly around it are four portions 7, which are relatively thin and capable of being broken through, to allow access of the liquid to the interior of the re-entrant portion 6. (It should be noted that, for the sake of simplicity, the re-entrant portions 6 and 9 below are not depicted on FIGS. 2-4).

Within the insert 4 there is firmly mounted a capillary-bearing member. This has basically the form of a cylindrical wall 8, which wall is closed at the bottom and has formed at this bottom a re-entrant portion 9 that matches in shape the re-entrant portion 6 of the wall 5 and is secured to it by means of a stud 10, such that the capillary-bearing member cannot move. The cylindrical wall 8 and the re-entrant portion 9 are made in a single piece of resilient plastics material.

Spaced equidistantly around the wall 8 and integral with it are four breaker elements 11 bearing capillary channels 12. These breaker elements and their associated capillary channels extend the entire length of the wall, and are attached to thereto by thinner connecting portions of the wall 13, capillary units and capillary bearing units being positioned against the four breakable portions 7 of the insert wall 5 and configured so that, in place, the breaker elements 11 are forced away from the breakable portions 7 against the resilience of the connecting portions 13. The breaker elements 11 additionally bear a projection 14, which assists in breaking through the breakable portions 7.

Lying concentrically within the cylindrical wall 8 is a further essentially cylindrical member 15. This is internally shaped so as to correspond to the shape of the re-entrant portion 9 and to be rotatable thereon, by means of turning a top 16. On its surface, it bears four equidistantly-spaced cams 17 that, on turning the top 16, will be brought simultaneously into contact with the breakable portions 7.

In operation (as is shown in FIG. 4), the rotation of the cylindrical member 15 causes the cams 17 to move to the breaker elements 11. These cams force the breaker elements 11 through the breakable wall portions 7, thus allowing liquid to flow through to the interior of the insert 4 and to the capillary channels 12, by which the liquid is transported to the atmosphere.

1. An apparatus for the dissemination of volatile liquid into an atmosphere, the apparatus comprising a reservoir containing volatile liquid and at least one liquid transfer member that is adapted to transfer liquid from the reservoir to the atmosphere, the transfer member being separated from the liquid, prior to putting the apparatus into operation, by a wall having at least one breakable section, there being present at least one breaker element that, when the apparatus is put into operation, is capable of breaking through the breakable section on being urged into contact therewith, thereby exposing the transfer member to the liquid.

2. An apparatus according to claim 1, in which the transfer member comprises capillary channels.

3. An apparatus according to claim 2, in which the capillary channels are incorporated in the breaker element.

4. An apparatus according to claim 1, in which the breaking of the breakable section by the breaker element is performed by rotation of part of the apparatus.

5. An apparatus according to claim 4, in which that part of the apparatus bearing the breaker element is rotatable with respect to the breakable section and there is positioned opposite the breakable section a stationary ramp adapted to urge the breaker element into breaking contact with the breakable section when the breaker element is rotated.

6. An apparatus according to claim 4, in which that part of the apparatus bearing the breaker element is stationary and is mounted in close proximity to the breakable section, the apparatus additionally comprising a rotatable member bearing a ramp adapted to urge the breaker element against and through the breakable section when the rotatable member is rotated.

7. A method for disseminating controllably from a reservoir into an atmosphere a volatile liquid by providing in the reservoir a transfer member adapted to convey liquid from the reservoir to the atmosphere, the transfer member being separated from the liquid by a wall in which there is provided at least one breakable section, and causing dissemination when desired by urging a breaker element capable of breaking through the breakable section into breaking contact with that section, thus exposing the transfer member to the liquid.

8. An apparatus according to claim 2, in which the breaking of the breakable section by the breaker element is performed by rotation of part of the apparatus.

9. An apparatus according to claim 8, in which that part of the apparatus bearing the breaker element is rotatable with respect to the breakable section and there is positioned opposite the breakable section a stationary ramp adapted to urge the breaker element into breaking contact with the breakable section when the breaker element is rotated.

10. An apparatus according to claim 8, in which that part of the apparatus bearing the breaker element is stationary and is mounted in close proximity to the breakable section, the apparatus additionally comprising a rotatable member bearing a ramp adapted to urge the breaker element against and through the breakable section when the rotatable member is rotated.
